(19) United States
(12) Patent Application Publication
(10) Pub. No.: US 2026/0306251 A1
(43) Pub. Date: Oct. 8, 2026

(54) INTRAOCULAR LENS SCAFFOLD DEVICE AND METHODS

(71) Applicant: Peter ZACHARIA, Worcester, MA (US)

(72) Inventor: Peter ZACHARIA, Worcester, MA (US)

(21) Appl. No.: 19/640,859

(22) Filed: Apr. 7, 2026

Related U.S. Application Data
(60) Provisional application No. 63/784,902, filed on Apr. 7, 2025.
(60) Provisional application No. 63/935,068, filed on Dec. 9, 2025.

(51) Int. Cl. A61F 2/16

(52) U.S. Cl. CPC A61F 2/1645; A61F 2002/169; A61F 2002/16902

(57) ABSTRACT

Disclosed are surgical devices, methods and procedures for implantation and employment of supports and/or scaffolding devices within a human and/or animal eye.

### CROSS-REFERENCE TO RELATED APPLICATIONS

This application claims the benefit of U.S. Provisional Application No. 63/784,902 entitled “INTRAOCULAR LENS SCAFFOLD DEVICE AND METHODS” filed Apr. 7, 2025, and U.S. Provisional Application No. 63/935,068 entitled “INTRAOCULAR LENS SCAFFOLD DEVICE AND METHODS” filed Dec. 9, 2025, the disclosures of which are each incorporated by reference herein in their entireties.

### FIELD OF THE INVENTION

The present technology relates to supports and/or scaffolding devices for implantation in the human and/or animal eye. Such devices can be useful for a variety of surgical procedures, including during prosthetic posterior chamber intraocular lens implantation in absence of support from natural lens capsule and zonule complex of the human eye.

### BACKGROUND AND SUMMARY OF THE INVENTION

The most desirable placement for a prosthetic intraocular lens after cataract surgery is in the space from which the natural lens or cataract has been removed, the area within the lens capsule after the lens material has been remove. Complications can occur either during cataract surgery or after cataract surgery which result in the absence of the lens capsule which is typically held in place by the zonules of the eye which project from the ciliary processes. The ciliary processes are projections of the ciliary body, the muscle within the eye responsible for accommodation, which induce a change in shape of the natural lens to allow for the eye to shift focus from distance to near. The ciliary body with its centripetally projecting ciliary processes is located directly posterior to the iris within the eyeball. Between the ciliary processes and the iris is a space termed the ciliary sulcus.

During cataract surgery, complications may occur which result in disruption of the lens capsule preventing placement of the intraocular lens within the capsular bag. If there is only partial disruption of the capsular bag structure, it may be possible to use the capsular bag as a scaffold for support of placement of the prosthetic posterior chamber lens. In such case the lens implant would typically rest anterior to but not within the capsular bag, but still posterior to the iris. Posterior chamber is the term for the area encompassing the space behind the iris and anterior to the natural lens and capsule, and also the space within the capsule itself. Support from a surgically disrupted lens capsule is typically deemed sufficient for support of a prosthetic posterior chamber lens when the peripheral capsule remains intact and connected to the ciliary body by the zonules for most of the circumference of the capsule—such that presence of this support will desirably prevent the prosthetic lens from falling posteriorly to the posterior chamber plane into the vitreous cavity. In such case, the term for placement of the posterior chamber lens implant is “sulcus placement” of the intraocular lens instead of “in the (capsular) bag placement.”

There are various reasons for preferential placement of a prosthetic lens within the plane of the posterior chamber besides simply mimicking natural anatomy, which can include better optical quality and prevention of complications arising from placement of a prosthetic lens in the alternate anterior chamber location—the space anterior to the iris. These complications can include the potential for injury to the cornea (the clear anterior wall of the eye through which light passes into the eye) as well as potential injury to the drainage system of the eye. This drainage system lies within the anterior chamber angle and includes a trabecular meshwork which lie at the very periphery of the iris within the anterior chamber. Injury to the cornea can result in opacification of the cornea which will reduce the quality of vision. Injury to the drainage structures of the eye may result in elevated intraocular pressure which can impair the vision permanently by causing glaucoma.

In addition to surgical complications during cataract surgery, other conditions may prevent placement of a prosthetic lens within the lens capsule—also termed the capsular bag. Eye diseases which weaken the zonules holding the lens and its capsule in place include diseases such as exfoliation syndrome, Marfan's Syndrome, and homocystinuria. Zonules may be disrupted from non - surgical trauma to the eye or even inadvertent trauma during eye surgery other than cataract surgery. These conditions may result in weakened or absent zonular support before cataract surgery when the natural lens is in place or long after cataract surgery when there may have already been uncomplicated placement of the prosthetic lens within the capsular bag. Subluxation (partial disruption of lens position) or dislocation (complete disruption of lens position) of the natural or prosthetic lens may occur in these conditions.

Prosthetic lenses typically have two main parts, the optic through which light passes and is refracted for focusing onto the retina, and the haptics which support the position of the optic by attaching on or resting on parts of the eye anatomy. Replacement of the natural or prosthetic lens in any of the situations when there is an absence of sufficient capsular support can be performed in different ways. A prosthetic intraocular lens can be placed in the anterior chamber as discussed above, in which case the haptics of the lens can be supported by the angle of the eye—the recess between the cornea and the iris—which is also the area which contains the drainage system of the eye. Prosthetic lenses placed in the posterior chamber of the eye in the absence of capsular support must be attached and have their position stabilized by various methods. The lenses can be attached to the posterior surface of the iris with sutures or to the sclera either with sutures or by placing the haptics directly within scleral tunnels created for support of the haptics. These methods can be successful but typically require above average technical skill and experience for an ophthalmic surgeon, and even in the hands of experienced surgeons can result in complications such as decentration or tilting of the prosthetic lens after placement. Iris sutured lenses can disrupt iris anatomy and prevent adequate dilation of the pupil during eye examinations. Lenses sutured to either iris or sclera can become displaced when sutures often dissolve or break years after placement. Intraocular lenses fixed in place with intrascleral tunnel haptic fixation can result in haptic erosion through the sclera and exposure through the outer surface of the sclera requiring removal or procedures to cover the haptics. Accordingly, there is need for further improvement in surgical placement of prosthetic intraocular lens implants, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

Various embodiments herein disclose methods and apparatus for placing a frame structure or “scaffolding” within a posterior chamber of a patient's eye to replace the support provided by the capsular bag and zonules when these structures may be absent and/or damaged, with the scaffolding supporting placement of a posterior chamber intraocular lens either after complicated cataract surgery or in situations where the natural or prosthetic lens implant may become subluxed (e.g., partially dislocated) or dislocated. The disclosed apparatus may function either without need for sutures to hold it in place or can also be sutured in place and facilitated by features included in the device for additional stability. One significant advantage of the disclosed system and apparatus is to desirably improve stability and centration and reduce the chance of tilting of the prosthetic posterior chamber lens when placed in the absence of support from the lens capsule and zonule complex. Another goal for the disclosed apparatus is to simplify placement of the posterior chamber lens implant when lens capsule and zonules are absent.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings.

FIG. 1 depicts a cross-sectional perspective view of an eye showing a naturally occurring capsular bag and zonules with the haptics and optic of an intraocular lens positioned therein;

FIG. 2 depicts a top plan view of one exemplary embodiment of a scaffold device constructed in accordance with various teachings of the present invention;

FIG. 3A depicts a rotated perspective view of the scaffold device of FIG. 2;

FIG. 3B depicts an enlarged view of a portion of the scaffold device of FIG. 3A;

FIG. 4 depicts the scaffold device of FIG. 1 holding a posterior chamber style intraocular lens;

FIG. 5 depicts a cross-sectional view of an eye with exemplary insertion of a delivery tube containing a scaffolding device through an incision made at the limbus;

FIG. 6 depicts a cross-sectional view of an eye with a scaffold device and intraocular lens in place;

FIG. 7 depicts an exemplary sizing gauge which allows for selection of an optimal size of scaffold device; and

FIG. 8 depicts another exemplary embodiment of a scaffold device with pre-attached sutures for fixation.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals may represent similar parts throughout the several views of the drawings. In addition, the following is a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In various embodiments, the terms “including,” “comprising” and variations thereof, as used in this disclosure, should be interpreted as “including, but not limited to,” unless expressly specified otherwise. The terms “a,” “an,” and “the,” as used in this disclosure, mean “one or more,” unless expressly specified otherwise.

In some embodiments, devices and/or device components that may be disclosed in communication with each other need not necessarily be in continuous communication with each other, unless expressly specified otherwise. In addition, components that are in direct contact with each other may contact each other directly or indirectly through one or more intermediary articles or devices. The device(s) disclosed herein may comprise various surgical materials well known by those skilled in the art.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single component, device and/or article is described herein, it should be readily apparent that more than one component, device and/or article may be used in place of a single component, device and/or article, unless otherwise noted. Similarly, where more than one component, device and/or article is described herein, it should be readily apparent that a single component, device and/or article may be used in place of the more than one component, device and/or article, unless otherwise noted. The functionality or the features of a component, device and/or article may be alternatively embodied by one or more other components, devices and/or articles which are not explicitly described as having such functionality or features.

Certain exemplary embodiments will now be described to provide an overall understanding of the principles of the structure, function, manufacture, and use of the components, devices and methods disclosed herein. One or more examples of these embodiments are illustrated in the accompanying drawings. Those of ordinary skill in the art will understand that the components, devices and/or methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides various components, devices, systems and methods for treating various anatomical structures of the eye of humans and/or other animals. While the disclosed embodiments may be particularly well suited for use during surgical procedures for the repair, fixation and/or support of an intraocular lens in the human eye, it should be understood that various other surgical procedures and/or anatomical locations of the body may benefit from various features of the present invention.

In accordance with various aspects of the present subject matter, various methods for manufacturing the device and/or associated components, including devices and/or implant sets within any of the details described with the present application are provided.

In at least one exemplary aspect, the invention further provides for devices and surgical methods for fixing an intraocular lens within a patient's eye where various naturally occurring support tissues such as the zonules and/or capsule/capsular remnants may be absent, damaged and/or insufficient in some manner.

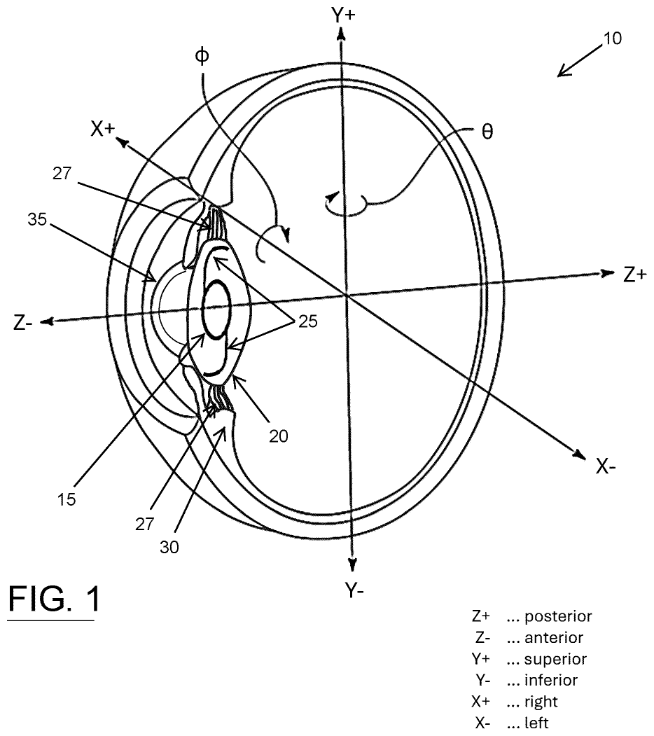

Fixation of an intraocular lens to the ciliary sulcus is a well accepted practice, where the haptics and optic of the intraocular lens can be rested on the zonule and capsule remnants (these prevent movement or tilting of the intraocular lens optic in the z-axis—see labelled axes in FIG. 1), wherein there desirably is a two point stabilization by the haptics extending into the sulcus, tending to prevent decentration of the optic within the plane of the intraocular lens (e.g., prevents decentration of the optic in the x or y axes).

However, where the zonules and capsule are absent, damaged and/or insufficient in some manner to provide a desired level of support, the disclosed scaffold device can be utilized to provide a 360-degree circumferential fixation of the intraocular lens within the ciliary sulcus, providing improved ciliary sulcus fixation as compared to the current two-point fixation techniques. The present invention can stabilize the intraocular lens in three ways: (1) the scaffold device desirably prevents posterior dislocation or tilting (e.g., along the z-axis—see labelled axes FIG. 1), (2) the scaffold device desirably prevents decentration within the plane of the lens (e.g., along the x and y axes—see labelled axes FIG. 1) by locking the intraocular lens in place, and (3) the scaffold device desirably inhibits or prevents rotation of the intraocular lens by locking the intraocular lens in place within the scaffold. Moreover, the disclosed scaffold device can desirably accomplish all of these objectives where remnants of the zonule and/or capsule are no longer present and/or useful.

Even where the zonules and capsule may be present, these structures may be disrupted enough so that decentration of the intraocular lens in the x or y axis or rotation of the intraocular lens cannot be prevented during simple sulcus placement. Such can occur in various subsets of complicated cataract surgery cases, even when remnants of the zonules and capsule may be sufficiently present to prevent posterior dislocation of an intraocular lens. In such situations, the disclosed scaffold device can have particular utility, and it can allow use of a wider variety of intraocular lenses such a toric or multifocal intraocular lenses, even when zonules and capsule are present but are anatomically disrupted.

The disclosed scaffold device will desirably allow greater versatility in a surgeon's choice of lens implants. The disclosed devices accommodate placement of a greater variety of posterior chamber lens implant products, while only a limited number of posterior chamber lens implants are currently available and/or suited for fixation in the posterior chamber with current techniques. Specific examples of this are toric and multifocal posterior chamber lenses, which can be more easily and reliably placed with the disclosed scaffold device. For example, multifocal lenses require precise antero-posterior axis positioning, while toric lenses require precise rotational axis alignment. Achieving these requirements is more difficult and less predictable with current techniques. With the disclosed scaffold device and related methods, a posterior chamber lens can be held in stable fashion in the same plane as typical intracapsular bag placement, and rotation of the lens with eye movement is inhibited and/or prevented. In addition, various features exist to prevent continuous movement or vibration of the lens implant, which can cause chafing of the iris and ciliary body and result in inflammatory conditions, bleeding, or glaucoma, a syndrome referred to as UGH (Uveitis, glaucoma, hyphema) syndrome.

In various embodiments, a scaffold device will desirably be capable of implantation in any eye, even those situations where the eye may lack the capsular bag and/or zonule complex typically employed for placement of a posterior chamber intraocular lens. A device capable of employing 360-degree fixation within the ciliary sulcus, to rest on the ciliary body shelf, and also which employs pressure against the outer wall of the sulcus or friction to achieve fixation and/or stability against movement or rotation, such as various embodiments described herein, can be highly useful in various anatomical situations. Moreover, disclosed embodiments which incorporate a fixation or “locking” mechanism to prevent unwanted rotation of the intraocular lens within the eye can be exceptionally useful during certain surgical procedures.

FIG. 1 shows a cross-section view of an eye 10 showing the ciliary body 30 and iris 35, with standard placement of a posterior chamber lens 15 within a capsular bag structure 20 after cataract surgery. The haptics 25 of the lens 15 desirably stabilize and maintain a central position of the optic portion of the lens within the capsular bag 20, with the capsular bag and zonules 27 present if they are not disrupted by surgical or other trauma or disease process. However, where the supporting anatomical structures are missing and/or damaged, such placement of a posterior chamber lens within the eye may be difficult and/or impossible to accomplish.

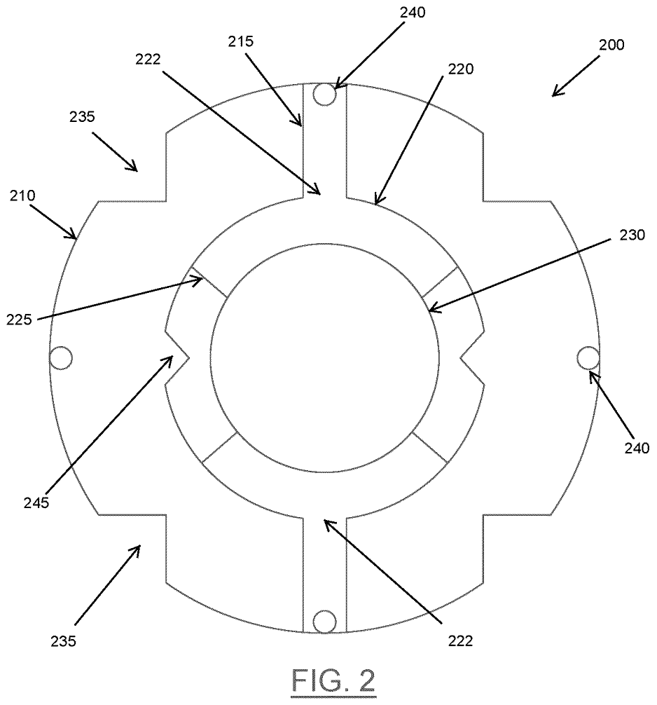

FIG. 2 depicts a top plan view of one exemplary embodiment of a scaffold device 200 constructed in accordance with various teachings of the present invention. In this embodiment, the device 200 comprises a biocompatible elastic and/or flexible wire or tubular frame which includes an outer ring 210, a middle ring 220 and an inner ring 230, with the outer and middle rings connected by outer connectors 215 and the middle and inner rings connected by inner connectors or crossbars 225. The outer ring 210 is shown with a plurality of kinked or bent sections 235 which allow the outer ring to bend, expand and/or flex in a desired and/or controlled manner as well as to provide circumferential flexibility and/or frictional stabilization to the outer wall of the ciliary sulcus after device implantation. Four loops 240 can be attached to or otherwise incorporated into the outer ring 210 to facilitate grasping and/or positioning of the device using surgical tools, as well as to provide a suture attachment location for optional fixation of the device 200 to surrounding anatomical structures. In this embodiment, the middle ring 200 is joined to the outer ring 210 by outer connectors 215 comprising four straight segments, wherein two pairs of the outer connectors are separated by a gap 222 which desirably allows for sliding and “tucking” of the intraocular lens haptics therethrough and posteriorly to the plane of the scaffold. If desired, these straight segments 215 may be angled or “tilted” posteriorly going from the plane of the outer ring to the plane of the middle and inner rings, to allow for more physiologic posterior placement of the intraocular lens relative to the scaffold device. If desired, the inner and/or intermediate ring(s) may be located within a plane posterior to the plane of the outer ring by posterior angulation of the spokes connecting the outer and intermediate rings, which can be a useful feature to prevent the lens implant from making unwanted contact with the iris in certain anatomical situations.

Optionally, bent expansion elements 245 can also be incorporated into the middle ring 220 to desirably allow for changes in circumference that may be needed during implantation and achieving a final resting configuration in the target anatomy. As shown, the inner ring 230 is attached to the middle ring 220 by straight bridging segments 225.

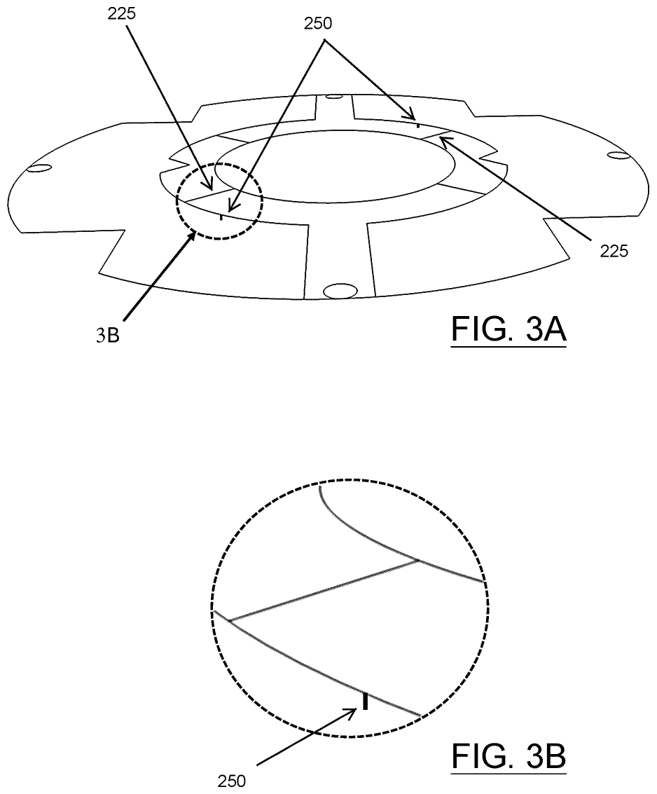

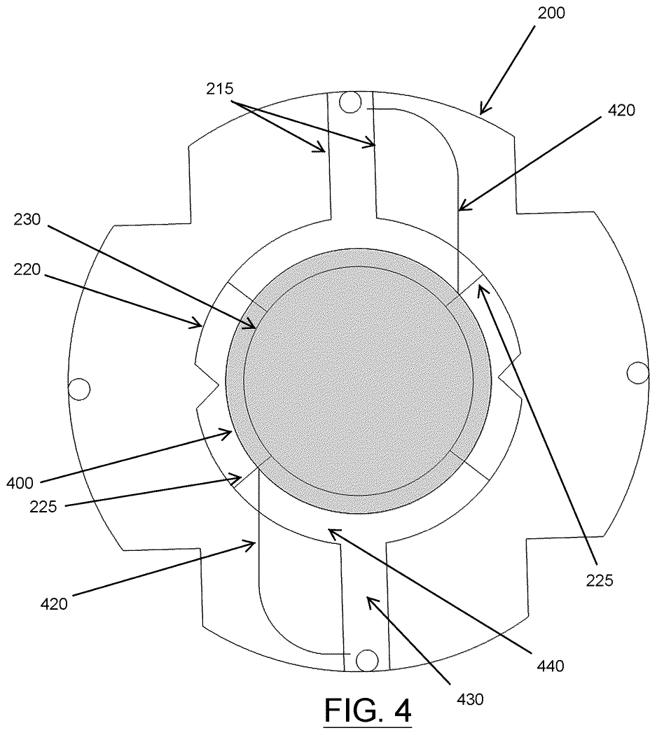

FIG. 3A depicts a slightly rotated perspective view of the scaffold device 200 with an upper end rotated into the page and a lower end rotated out of the page relative to the view of FIG. 2. This view shows similar structures as described in FIG. 2, but additionally depicts two small projections 250 extending posteriorly from the middle ring 220, with an enlarged view of the projections proved in FIG. 3B. These projections 250 will desirably allow for the haptics to be locked in place when the haptics are placed posterior to the plane of the optic, trapping the haptics of the intraocular lens (not shown) against and/or between each projection 250 and an adjacent crossbar 225 upon clockwise rotation of the intraocular lens during implantation. This arrangement desirably secures the intraocular lens in 3 dimensions against movement and prevent rotation of the lens, which can be best seen in FIG. 4.

The projections in FIG. 3B are depicted as located diametrically opposite or 180 degrees from each other to accommodate capture of the haptics, which can be captured in a rachet like fashion between these posterior projections and the crossbars as the prosthetic lens is rotated clockwise during implantation, other arrangements of the projections are contemplated herein. The flexibility of standard intraocular lens haptics desirably allows for slight flexion of the haptics during implantation to allow for this movement and/or positioning. If desired, the projections may extend perpendicularly from the middle ring, or alternatively the projections might be angled away from and/or towards the direction of lens rotation so as to allow the haptics to flex around a slanted and/or detent surface and subsequently be captured within a notched or acute surface of the projection.

In various embodiments, the middle ring 220 can include two gaps 222 in continuity along the circumference of the middle ring, with the middle ring connected to the outer and inner rings with a plurality of linear supporting segment. The two gaps 222 can allow the haptics to be tucked behind the plane of the optic and portions of the scaffold during insertion of the lens. Securement of the lens can prevent undesirable antero-posterior movement, rotation, or any shifting in position of the prosthetic lens relative to the scaffold and/or eye structures. Such an arrangement can also desirably prevent intermittent and/or unwanted contact between the haptics and the posterior surface of the iris or ciliary body, which might cause chafing during continued movement of haptics (which can cause inflammatory conditions, bleeding, or glaucoma). The middle ring may also optionally incorporate bent expansion elements 245 within the circumference as needed for expansion and contraction to facilitate placement during surgical implantation, and also to prevent distortion and nonplanar configuration of the ring itself and the linear supports once the device is positioned in the ciliary sulcus.

FIG. 4 depicts an anterior view of the scaffold device 200 holding an associated intraocular lens 400, with the intraocular lens positioned against and engaged with the scaffold device 200. Desirably, the optic 410 of the intraocular lens 400 will resting adjacent to and against anterior surfaces of the inner ring 230 and middle ring 220 of the scaffold 200, with the haptics 420 positioned posterior to the middle ring 220 while being prevented from rotating back counterclockwise by the presence of the posterior projections 250 (see FIGS. 3A and 3B) projecting posteriorly from the middle ring 220, and also being prevented from rotating further clockwise by the presence of the crossbars 225. Optimally, the device 200 can secure and hold a posterior chamber style intraocular lens with the optic 410 resting anterior to the device 200 and centered within the device 200, with the haptics 420 positioned posterior to various portions of the device 200 such as the middle ring 220 and optionally the straight segments 215.

In various alternative embodiments, the outer connectors 215 between the outer ring and the middle ring may be angulated slightly posteriorly so that the middle and inner rings are in a plane slightly posterior to the plane of the outer ring and/or ciliary sulcus. This could potentially allow for a small gap between the IOL optic and the iris to enable a more physiologic positioning and to reduce chances of chafing of the posterior iris by the edge of the optic. In other alternative embodiments, one or more of the cross bars 225 between the middle ring and the inner ring may be positioned flush and/or angled relative to the middle ring and/or the inner ring, such that the inner and/or middle rings may be offset relative to each other.

The employment of gaps in the scaffold device desirably allows for posterior displacement of the lens haptics, which can accommodate a wide variety of commercially available posterior chamber intraocular lens designs. This design feature can also allow for the use of widely available single piece lens designs, such as single piece acrylic posterior chamber lenses. In many cases, existing single piece posterior chamber lenses can be unsafe for use in sulcus implantation using existing surgical techniques as the haptics of these lenses are thicker and incorporate sharp edges which can chafe the ciliary body and/or posterior surface of the iris, resulting in the creation or exacerbation of inflammatory conditions, bleeding, or glaucoma, including a condition called UGH (uveitis, glaucoma, hyphema) syndrome. Posterior displacement of the lens haptics desirably inhibits and/or prevents such conditions from occurring and/or worsening. Moreover, enabling use of single piece intraocular lens designs would make available for patient use the more widely available features of multifocal and toric lens designs currently produced using the single piece intraocular lens platforms.

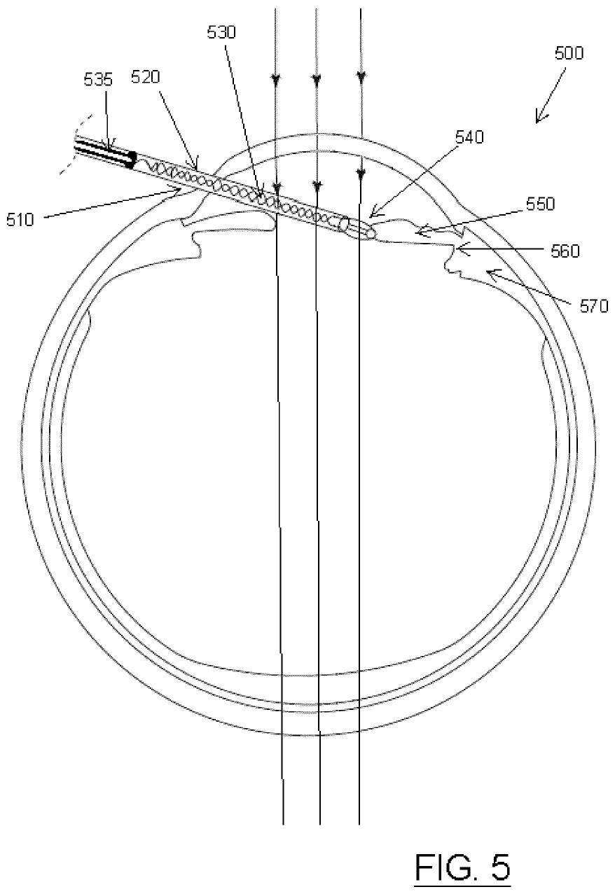

FIG. 5 depicts a cross-sectional view of an eye 500 with an incision made at the limbus 510 and insertion of a delivery tube 520 containing a scaffold device 530 with a plunger 535 advancing the device 530 and a distal tip 540 of the scaffold device leaving the tube to exit beneath an iris 550 and to be guided to the ciliary sulcus 560 and to rest upon the shelf provided by the ciliary processes of the ciliary body 570. If desired, the entire device assembly can be placed within the eye by injection through the delivery tube with an external diameter sufficiently small to place within a limbal or scleral tunnel incision made for small incision cataract surgery, in most cases between 2 and 3 mm.

For the process of lens implantation in an eye lacking capsular support, the scaffold device 200 can desirably be first inserted into the eye and stabilized behind the iris. Following this, a posterior chamber style lens can be inserted into the eye, positioned anteriorly to the scaffold, with each of the haptics 420 guided posteriorly through a slot 430 between the straight segments 215 and a slot 440 between the inner ring and the middle ring 220, wherein the lens 400 can be rotated slightly clockwise until the haptics 420 are engaged past the projections 250 and crossbars 225, which desirably provides stability of the lens position in three dimensions and prevents unwanted rotation of the optic.

The internal ring 230 can desirably have a diameter small enough to support the edges of an intraocular lens prosthesis being implanted. Typical IOL diameters are between 6 and 7 mm, so one exemplary embodiment of the internal ring could be 0.5 to 1 mm smaller in diameter than the intraocular lens optic, to desirably allow the edges of the optic to rest on the internal ring surface. In some embodiments, a diameter of 5.5 mm could be optimal for production of a universally compatible device with respect to size of lens optic in general use. The scaffold device as a whole may be produced with different dimensions of the various components such as internal ring to accommodate different intraocular lenses with differing optic diameters and haptic features such as haptic angulation with respect to the optic. However, the overall configuration of the device can alternatively allow for a single standard dimension, such as a 5.5 mm inner ring, to accommodate the majority of commercially available posterior chamber lenses.

The device can be constructed from a medically suitable wire or tubular material which provides sufficient flexibility for the device to roll up or fold within a deployment tube for insertion and/or injection into an anterior chamber, and which would desirably retain shape memory. Non-magnetic materials such as Prolene or Nitinol can be used, as desired.

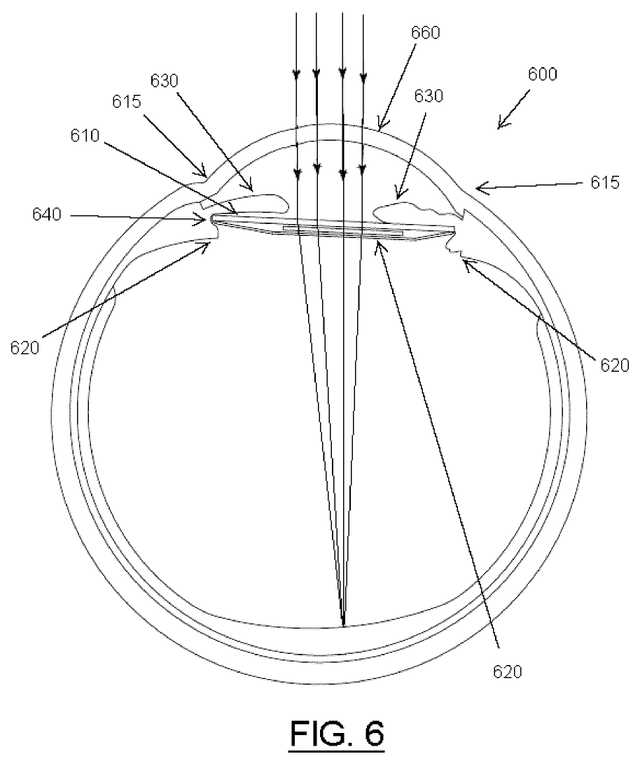

FIG. 6 depicts a cross-sectional view of an eye 600 with a scaffold device 610 and intraocular lens 620 in place. An outer ring of the scaffold 610 is placed anterior to the ciliary processes 620 and posterior to the iris 630 in the space called the ciliary sulcus 640. This outer ring will desirably occupy the entire circumference of the sulcus, such that the entire circumference of the sulcus and the posterior shelf of ciliary body would provide adequate support to maintain positional stability. This external ring incorporates kinked or bent sections 235 (e.g., expansion elements), which in some embodiments can comprise V-shaped relaxing segments, allowing collapse of the circumference of the ring during placement and then restoration of larger circumference after positioning to provide a centrifugally directed force after placement of the ring within the ciliary sulcus to prevent dislocation or movement. One exemplary representation of these expansion elements can be a simple V-shape as drawn which can allow the ring to collapse and expand. The ring could be produced in a variety of sizes with the optimal sizing for an individual eye being a diameter slightly greater than that of the ciliary sulcus. The number and sizing of these expansion elements may vary but in most cases four elements spaced evenly along the circumference of the ring will desirably be sufficient to allow for the contraction and expansion necessary during placement. As previously noted, loops 240 can be incorporated into the outer ring to facilitate positioning and grasping of the scaffold device using surgical microhooks during surgery and also to allow for suturing the device to the sclera if desired.

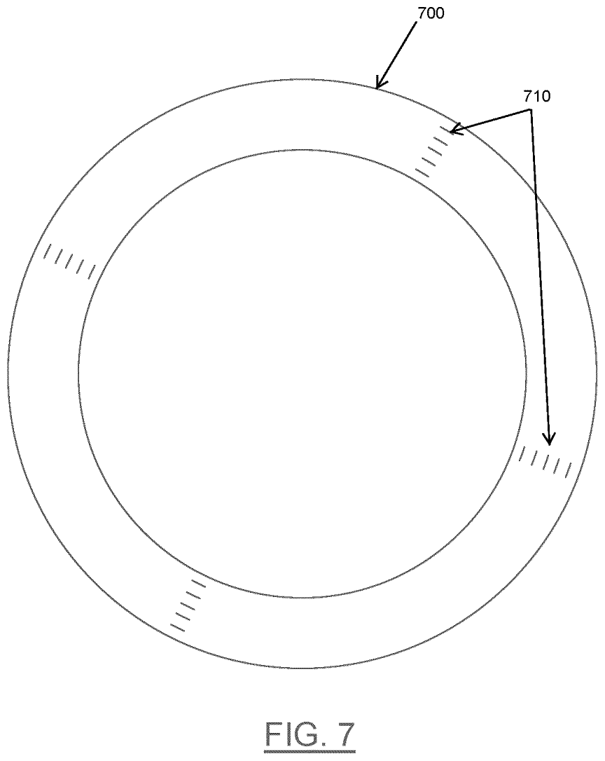

FIG. 7 depicts one exemplary embodiment of a sizing gauge 700 which could be shaped in the form of a frustum of a sphere to determine proper size of scaffold to be used. If desired, the sizing gauge may be provided in differing sizes. The guide 700 could be placed on an eye prior to surgery, where it may encircle the limbus 615 of the cornea 660. The guide 700 may include millimeter or other markings 710 etched into the surface. Such a tool could assist the surgeon with choosing the proper diameter scaffold device to be used, although a large variety of scaffold diameters may not be required because of the significant variability in final resting diameters enabled by the expansion elements of the scaffold, which can accommodate a wide range of shapes and/or sizes of eye anatomy. However, if a wider range of scaffold devices are available for use, the millimeter or other markings can allow for selection of an optimal size, shape and/or configuration of scaffold. For example, the markings could facilitate estimation of the diameter of the ciliary sulcus for which a scaffold with maximally expanded diameter slightly greater than diameter of the sulcus might be selected.

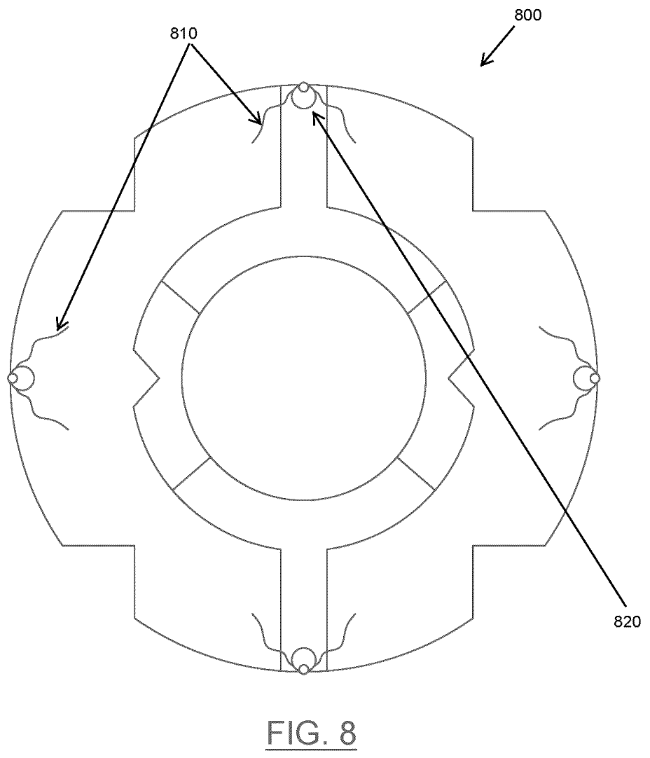

FIG. 8 depicts another exemplary embodiment of a scaffold device 800. In this embodiment, the device can be provided with pre-attached sutures 810 for fixation. Such an embodiment may be particularly useful to surgeons who choose to fixate the device to sclera with sutures. A needle could be used to make two scleral perforations in the area of each suture 810 and loop 820, such as two scleral perforations with a needle above and below the ring from the outside of the sclera and/or with one anterior perforation and another posterior perforation made to the plane of the outer ring in the ciliary sulcus. The sutures could then be externalized with an instrument and tied external to the sclera for additional stability if determined necessary by the surgeon. Various other embodiments of the device may be available with 2 to 4 retention sutures attached to the inner aspect of the external ring at loops incorporated into the outer ring. Each end of the suture (e.g., one anterior and one posterior to the ring) could be externalized through the two perforations, whereupon the ends could be tied to stabilize the ring if the surgeon prefers to secure the ring with retention sutures.

In various embodiments, the scaffold devices described herein will allow for accurate placement of a toric intraocular lens prosthesis to correct astigmatism. In such case, the scaffold device could be rotated in its entirety along the ciliary sulcus to align the axis of the toric lens implant with the appropriate axis necessary to correct the patient's astigmatism, if such rotation were accomplished after placement of the intraocular lens onto the scaffold device.

In various alternative embodiments, the external ring may contain a fluorescent/reflective material or incorporate a light emitting feature which can be activated temporarily to enable visualization of position through sclera. This would permit confirmation of positioning along the entire circumference of sclera external to the ciliary sulcus. In addition, such a feature could facilitate suture reinforcement of the external ring to sclera by showing positioning of the ring. Fluorescent/reflective material would desirably become visible when the operating room is illuminated with blue light. If a light emitting feature is incorporated, packaging may incorporate a charging device to charge a capacitor contained within the external ring which would provide power to illuminate the light emitting feature for a limited time during surgery.

In some alternative embodiments, the outer ring of the scaffold device might incorporate a sensor to detect electrophysiologic activity within the ciliary body which could manifest when a patient attempts to accommodate as the patient may have done when the patient's natural lens was present in attempt to visualize an object or print held near to the eye by the patient. In such way this can function within a transduction mechanism to induce the supported lens implant to change refractive index or anteroposterior position or thickness to allow the patient to shift focus from distance to near. The placement of sensor and transduction mechanism(s) within the device can allow for accommodation or change in focus from far to near which may be useful in various anatomical situations.

If desired, the dimensions and/or design(s) of a single scaffold device can be selected to accommodate a wide majority of eye shapes and/or sizes, or a plurality of different sizes And/or shapes can be provided to accommodate different size ranges of eyes or to better accommodate different IOL types and compositions. For example, wider gaps 430 in a middle ring of a scaffold device or a variation in positioning in the posterior projections 250 might facilitate use of certain single piece acrylic intraocular lenses with haptics which are thicker or which may be angulated differently with respect to the lens optic. Such single piece acrylic lenses are currently indicated for in the bag placement only since the haptics may chafe the posterior surface of the iris, but the disclosed devices may allow for their use as discussed herein.

If desired, a “kit” or other packaging could include various components of differing shapes and/or sizes to accommodate different sizes and/or shapes of eye anatomy. Similarly, different components and/or portions thereof could comprise the same or a variety of different materials.

In accordance other aspects of the present subject matter, various methods for manufacturing implants and/or components thereof, as set for within any of the details described with the present application, are part of the scope of the present application.

While embodiments and applications of the present subject matter have been shown and described, it would be apparent that other embodiments, applications and aspects are possible and are thus contemplated and are within the scope of this application.

Note that, in various alternative embodiments, variations in the position and/or relationships between the various figures and/or modular components are contemplated, such that different relative positions of the various modules and/or component parts, depending upon specific module design and/or interchangeability, may be possible. In other words, different relative adjustment positions of the various components may be accomplished via adjustment in separation and/or surface angulation of one of more of the components to achieve a variety of resulting implant configurations, shapes and/or sizes, thereby accommodating virtually any expected anatomical variation.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An implantable support or scaffold for a human eye, comprising:
a biocompatible elastic outer ring having a first plurality of expansion elements;
an intermediate ring with a plurality of gaps in continuity along the circumference of the ring and at least two projections extending from the posterior aspect of the ring; and
an inner ring having an open center;
wherein the intermediate ring is spaced apart from the biocompatible elastic outer ring and is connected to the biocompatible elastic outer ring by a first plurality of spokes, and the intermediate ring is spaced apart from the inner ring and is connected to the inner ring by a second plurality of spokes, the first plurality of spokes being radially spaced apart from the second plurality of spokes.

2. The implantable support or scaffold for a human eye of claim 1, wherein the intermediate ring further comprises a second plurality of expansion elements, the first plurality of expansion elements being radially spaced apart from the second plurality of expansion elements.

3. The implantable support or scaffold for a human eye of claim 1, comprising a plurality of loops attached to the outer ring to facilitate suture attachment to the outer ring.

4. The implantable support or scaffold for a human eye of claim 1, wherein the outer ring comprises a fluorescent material.

5. The implantable support or scaffold for a human eye of claim 1, wherein the outer ring comprises a sensor or transduction mechanism which accommodates a change in focus from far to near.

6. An implantable support or scaffold for a human eye, comprising:
a biocompatible elastic outer ring having a first plurality of expansion elements;
an intermediate ring with a plurality of gaps in continuity along the circumference of the ring and at least two projections extending from the posterior aspect of the ring; and
an inner ring having an open center;
wherein the intermediate ring is spaced apart from the biocompatible elastic outer ring as is located within a plane posterior to the plane of the outer ring by a first plurality of angled spokes, and the intermediate ring is spaced apart from the inner ring and is connected to the inner ring by a second plurality of spokes, the first plurality of angled spokes being radially spaced apart from the second plurality of spokes.

7. The implantable support or scaffold for a human eye of claim 6, wherein at least a portion of the support rests on the ciliary body shelf and employs pressure against the outer wall of the sulcus to reduce and/or eliminate movement or rotation of the support relative to the human eye.

8. The implantable support or scaffold for a human eye of claim 6, wherein the support further comprises a locking mechanism to inhibit rotation or otherwise disassociation from the scaffold in any axis of the intraocular lens relative to the scaffold to the human eye.

9. A method of improving a patient's vision, comprising:
accessing an interior space of an eye;
advancing a support or scaffold into the interior space, the support or scaffold comprising a plurality of ring sections of differing sizes, at least two of the plurality of ring sections connected by a plurality of spokes;
coupling the support or scaffold with an inner periphery of the eye;
advancing an optic into the interior space, the optic comprising an optical zone, an outer periphery and a plurality of haptics; and
attaching the optic to the support or scaffold using the plurality of haptics.

10. The method of claim 9, wherein the step of advancing the optic into the interior space occurs after the step of advancing the support or scaffold into the interior space of the eye.

11. The method of claim 9, further comprising rotating the support or scaffold and attached optic to align an axis of the optic with an optimal axis necessary to correct an astigmatism of the patient's vision.

12. The method of claim 11, wherein the optic is a toric lens implant.

13. The method of claim 9, wherein the step of coupling the support or scaffold with an inner periphery of the eye comprises placing an outer ring section of the plurality of ring sections in contact with an outer wall of a ciliary sulcus of the eye.

14. The method of claim 9, wherein the step of coupling the support or scaffold with an inner periphery of the eye comprises suturing an outer ring section of the plurality of ring sections to a sclera of the eye.

15. The method of claim 9, wherein the plurality of haptics of the optic are not directly attached to the inner periphery of the eye.

16. The method of claim 9, wherein the support or scaffold is foldable.

17. The method of claim 9, further comprising the step of removing at least a portion of a natural lens capsule or natural zonule complex from the interior space of the eye.
